(19) United States
(12) Patent Application Publication
(10) Pub. No.: US 2020/0179706 A1
(43) Pub. Date: Jun. 11, 2020

(54) HEMODYNAMICALLY OPTIMIZED RATE RESPONSE PACING USING HEART SOUNDS

(71) Applicant: Cardiac Pacemakers, Inc., St. Paul, MN (US)

(72) Inventors: Pramodsingh Hirasingh Thakur, Woodbury, MN (US); James O. Gilkerson, Stillwater, MN (US); Ramesh Wariar, Blaine, MN (US); Qi An, Blaine, MN (US)

(21) Appl. No.: 16/701,919

(22) Filed: Dec. 3, 2019

Related U.S. Application Data
(60) Provisional application No. 62/775,760, filed on Dec. 5, 2018.

(51) Int. Cl. A61N 1/365; A61N 1/37; A61B 5/024; A61B 5/00

(52) U.S. Cl. CPC A61N 1/36592; A61N 1/3702; A61N 1/37512; A61B 5/742; A61B 5/024

(57) ABSTRACT

An apparatus comprises a stimulus circuit configured to deliver electrical pacing therapy to a subject when operatively coupled to a plurality of electrodes, a heart sound sensing circuit configured to produce a sensed heart sound signal representative of mechanical cardiac activity, a control circuit operatively coupled to the stimulus circuit and configured to vary a paced heart rate of the subject according to a specified paced heart rate protocol, and a signal processing circuit. The signal processing circuit is configured to obtain measures of a heart sound parameter as a function of heart rate using the sensed heart sound signal and determine a critical heart rate for the subject using the obtained measures of the heart sound parameter.

### CLAIM OF PRIORITY

This application claims the benefit of priority under 35 U.S.C. § 119(e) of U.S. Provisional Patent Application Ser. No. 62/775,760, filed on Dec. 5, 2018, which is herein incorporated by reference in its entirety.

### BACKGROUND

Ambulatory medical devices include implantable medical devices (IMDs), wearable medical devices, handheld medical devices, and other medical devices. Some examples of IMDs include cardiac function management (CFM) devices such as implantable pacemakers, implantable cardioverter defibrillators (ICDs), subcutaneous implantable cardioverter defibrillators (S-ICDs), cardiac resynchronization therapy devices (CRTs), and devices that include a combination of such capabilities. The devices can be used to treat patients or subjects using electrical or other therapy, or to aid a physician or caregiver in patient diagnosis through internal monitoring of a patient's condition.

Some implantable medical devices can be diagnostic-only devices, such as implantable loop recorders (ILRs), subcutaneously insertable cardiac monitors (ICMs), and subcutaneously insertable heart failure monitors (SubQ HFMs). The devices may include electrodes in communication with one or more sense amplifiers to monitor electrical heart activity within a patient or can include one or more sensors to monitor one or more other patient parameters. Subcutaneously implantable devices may include electrodes that are able to sense cardiac signals without being in direct contact with the patient's heart. Other examples of IMDs include implantable drug delivery systems or implantable devices with neural stimulation capability (e.g., vagus nerve stimulator, baroreflex stimulator, carotid sinus stimulator, spinal cord stimulator, deep brain stimulator, etc.).

Some examples of wearable medical devices include wearable cardioverter defibrillators (WCDs) and wearable diagnostic devices (e.g., an ambulatory monitoring vest, holter monitor, cardiac event monitor, or mobile cardiac telemetry devices). WCDs can be monitoring devices that include surface electrodes. The surface electrodes may be arranged to provide one or both of monitoring to provide surface electrocardiograms (ECGs) and delivery of cardioverter and defibrillator shock therapy. In some examples, a wearable medical device can also include a monitoring patch worn by the patient such as an adherable patch or can be included with an article of clothing worn by the patient.

Some examples of handheld medical devices include smartphones and personal data assistants (PDAs). The handheld devices can be diagnostic devices that record an electrocardiograph (ECG) or other physiological parameter while the device is resting in the patient's hand or being held to the patient's chest.

Some medical devices include one or more sensors to monitor different physiologic aspects of the patient. For example, the devices may derive measurements associated with a cardiac depolarization of the patient. Such monitoring devices can be implantable or wearable and the measurements can provide useful information concerning the cardiac health of the patient.

By monitoring cardiac signals indicative of expansions or contractions, IMDs can detect abnormally slow heart rate, or bradycardia. In response to an abnormally slow heart rate some CFM devices deliver electrical pacing stimulation energy to induce cardiac depolarization and contraction. The pacing stimulation energy is delivered to provide a depolarization rate that improves hemodynamic function of the patient. Delivery of pacing therapy should be optimized to ensure optimum therapy delivery and yet avoid unnecessary stress on the heart. However, strategies to optimally program pacing configurations are often developed heuristically or the programming for the pacing can be ad-hoc and only address one aspect of the patient's cardiac disease.

### OVERVIEW

This document relates generally to systems, devices, and methods that provide electrical pacing therapy to the heart of a patient or subject. In particular it relates to, systems, devices, and methods that determine a critical heart rate that optimizes pacing therapy. The device-determined critical heart rate can provide an upper bound or a maximum heart rate limit customized to a particular patient. This can improve the patient's experience with a prescribed medical device that delivers electrical pacing therapy.

Example 1 includes subject matter (such as an apparatus) comprising a stimulus circuit configured to deliver electrical pacing therapy to a subject when operatively coupled to a plurality of electrodes, a heart sound sensing circuit configured to produce a sensed heart sound signal representative of mechanical cardiac activity, and a signal processing circuit. The signal processing circuit is configured to obtain measures of a heart sound parameter as a function of heart rate using the sensed heart sound signal, determine a maximum pacing using a critical heart rate identified using the obtained measures of the heart sound parameter as a function of heart rate.

In Example 2, the subject matter of Example 1 optionally includes a signal processing circuit configured to obtain measures of S1 heart sound amplitude as the heart sound parameter; and identify the critical heart rate as a heart rate corresponding to a maximum measured S1 heart sound amplitude.

In Example 3, the subject matter of one or both of Examples 1 and 2 optionally includes a cardiac signal sensing circuit configured to produce a sensed cardiac signal representative of cardiac depolarization of the subject when coupled electrically to the plurality of electrodes; wherein signal processing circuit is configured to: obtain measures of a systolic time interval (STI) ratio as the heart sound parameter using the sensed cardiac signal and the sensed heart sound signal; and identify the critical heart rate as a heart rate corresponding to a minimum measured STI ratio.

In Example 4, the subject matter of Example 3 optionally includes a signal processing circuit configured to calculate, as the STI ratio, a ratio including a time interval between a cardiac signal fiducial and the S1 heart sound and a time interval between the S1 heart sound and an S2 heart sound.

In Example 5, the subject matter of one or any combination of Examples 1-4 optionally includes a signal processing circuit configured to: determine slope of the measures of the heart sound parameter as a function of heart rate; and identify the critical heart rate as a heart rate corresponding to a minimum determined slope.

In Example 6, the subject matter of one or any combination of Examples 1-5 optionally includes a control circuit operatively coupled to the stimulus circuit and configured to vary a paced heart rate of the subject according to a specified paced heart rate protocol. The signal processing is configured to collect the measures of the heart sound parameter according to the specified paced heart rate protocol and determine the critical heart rate using the collected measures of the heart sound parameter.

In Example 7, the subject matter of Example 6 optionally includes a control circuit configured to vary the paced heart rate according to one of a ramp-up pacing rate protocol or a pseudo-random pacing rate protocol.

In Example 8, the subject matter of one or both of Examples 6 and 7 optionally include a physiologic sensor circuit configured to produce a sensed physiological signal representative of physiological information of a subject; and a control circuit configured to change the paced heart rate up to a maximum heart rate limit according to the sensed physiological signal and set the maximum heart rate limit to the determined critical heart rate.

In Example 9, the subject matter of one or any combination of Examples 1-8 optionally includes a signal processing circuit configured to gather paired measures of the heart sound parameter and heart rate during naturally occurring changes in heart rate over a specified time interval and use the paired measures of the heart sound parameter and heart rate to determine the critical heart rate.

In Example 10, the subject matter of one or any combination of Examples 1-9 optionally includes a signal processing circuit configured to filter measures of the heart sound parameter after a change in heart rate and use filtered measures of the heart sound parameter as a function of heart rate to determine the critical rate

Example 11 includes subject matter (such as an automated method of operation a medical device, or a computer readable storage medium including instructions that when performed by the medical device cause the medical device to perform the method), or can optionally be combined with one or any combination of Examples 1-10 to include such subject matter, comprising sensing a heart sound signal representative of mechanical cardiac activity of a subject; delivering electrical cardiac pacing therapy to the subject, including varying a paced heart rate of the subject according to a specified paced heart rate protocol; obtaining measures of a heart sound parameter as a function of different paced heart rates using the sensed heart sound signal; and configuring a maximum pacing rate using a critical heart rate identified using the obtained measures of the heart sound parameter.

In Example 12, the subject matter of Example 11 optionally includes obtaining, as the heart sound parameter, measures of S1 heart sound amplitude as the paced heart rate is varied and identifying the critical heart rate as a heart rate corresponding to a maximum measured S1 heart sound amplitude.

In Example 13, the subject matter of one or both of Examples 11 and 12 optionally include producing a sensed cardiac signal representative of cardiac depolarization of the subject, obtaining, as the heart sound parameter, measures of a systolic time interval (STI) ratio using the sensed cardiac signal and the sensed heart sound signal as the paced rate is varied, and identifying the critical heart rate as a heart rate corresponding to a minimum measured STI ratio.

In Example 14, the subject matter of Example 13 optionally includes calculating a ratio including a time interval between a cardiac signal fiducial and the S1 heart sound and a time interval between the S1 heart sound and an S2 heart sound.

In Example 15, the subject matter of one or any combination of Examples 11-14 optionally includes determining a slope of the measures of the heart sound parameter as a function of heart rate and identifying the critical heart rate as a heart rate corresponding to a minimum determined slope.

Example 16 includes subject matter (such as an apparatus) comprising a stimulus circuit configured to deliver electrical pacing therapy to a subject when operatively coupled to a plurality of electrodes, a heart sound sensing circuit configured to produce a sensed heart sound signal representative of mechanical cardiac activity, and a signal processing circuit. The signal processing circuit is configured to obtain measures of a heart sound parameter as a function of heart rate using the sensed heart sound signal, and determine a maximum pacing rate using a critical heart rate identified using the obtained measures of the heart sound parameter as a function of heart rate.

In Example 17, the subject matter of Example 16 optionally includes a signal processing circuit configured to obtain measures of S1 heart sound amplitude as the heart sound parameter; and identify the critical heart rate as a heart rate corresponding to a maximum measured S1 heart sound amplitude.

In Example 18, the subject matter of one or both of Examples 16 and 17 optionally includes a cardiac signal sensing circuit configured to produce a sensed cardiac signal representative of cardiac depolarization of the subject when coupled electrically to the plurality of electrodes; wherein signal processing circuit is configured to: obtain measures of a systolic time interval (STI) ratio as the heart sound parameter using the sensed cardiac signal and the sensed heart sound signal; and identify the critical heart rate as a heart rate corresponding to a minimum measured STI ratio.

In Example 19, the subject matter of Example 18 optionally includes a signal processing circuit configured to calculate, as the STI ratio, a ratio including a time interval between a cardiac signal fiducial and the S1 heart sound and a time interval between the S1 heart sound and an S2 heart sound.

In Example 20, the subject matter of one or any combination of Examples 16-19 optionally includes a signal processing circuit configured to: determine slope of the measures of the heart sound parameter as a function of heart rate; and identify the critical heart rate as a heart rate corresponding to a minimum determined slope.

In Example 21, the subject matter of one or any combination of Examples 16-20 optionally includes a control circuit operatively coupled to the stimulus circuit and configured to vary a paced heart rate of the subject according to a specified paced heart rate protocol. The signal processing is configured to collect the measures of the heart sound parameter according to the specified paced heart rate protocol and determine the critical heart rate using the collected measures of the heart sound parameter.

In Example 22, the subject matter of Example 21 optionally includes a control circuit configured to vary the paced heart rate according to one of a ramp-up pacing rate protocol or a pseudo-random pacing rate protocol.

In Example 23, the subject matter of one or both of Examples 21 and 22 optionally include a physiologic sensor circuit configured to produce a sensed physiological signal representative of physiological information of a subject; and a control circuit configured to change the paced heart rate up to a maximum heart rate limit according to the sensed physiological signal and set the maximum heart rate limit to the determined critical heart rate.

In Example 24, the subject matter of one or any combination of Examples 16-23 optionally includes a signal processing circuit configured to gather paired measures of the heart sound parameter and heart rate during naturally occurring changes in heart rate over a specified time interval and use the paired measures of the heart sound parameter and heart rate to determine the critical heart rate.

In Example 25, the subject matter of one or any combination of Examples 16-24 optionally includes a signal processing circuit configured to filter measures of the heart sound parameter after a change in heart rate and use filtered measures of the heart sound parameter as a function of heart rate to determine the critical rate

Example 26 includes subject matter (such as an automated method of operation a medical device, or a computer readable storage medium including instructions that when performed by the medical device cause the medical device to perform the method), or can optionally be combined with one or any combination of Examples 16-26 to include such subject matter, comprising sensing a heart sound signal representative of mechanical cardiac activity of a subject; delivering electrical cardiac pacing therapy to the subject, including varying a paced heart rate of the subject according to a specified paced heart rate protocol; obtaining measures of a heart sound parameter as a function of different paced heart rates using the sensed heart sound signal; obtaining measures of a heart sound parameter as a function of different paced heart rates using the sensed heart sound signal; and configuring a maximum pacing rate using a critical heart rate identified using the obtained measures of the heart sound parameter.

In Example 27, the subject matter of Example 26 optionally includes obtaining, as the heart sound parameter, measures of S1 heart sound amplitude as the paced heart rate is varied and identifying the critical heart rate as a heart rate corresponding to a maximum measured S1 heart sound amplitude.

In Example 28, the subject matter of one or both of Examples 26 and 27 optionally include producing a sensed cardiac signal representative of cardiac depolarization of the subject, obtaining, as the heart sound parameter, measures of a systolic time interval (STI) ratio using the sensed cardiac signal and the sensed heart sound signal as the paced rate is varied, and identifying the critical heart rate as a heart rate corresponding to a minimum measured STI ratio.

In Example 29, the subject matter of Example 28 optionally includes calculating a ratio including a time interval between a cardiac signal fiducial and the S1 heart sound and a time interval between the S1 heart sound and an S2 heart sound.

In Example 30, the subject matter of one or any combination of Examples 26-29 optionally includes determining a slope of the measures of the heart sound parameter as a function of heart rate and identifying the critical heart rate as a heart rate corresponding to a minimum determined slope.

Example 31 can include subject matter (such as a medical device system) or can optionally be combined with one or any combination of Examples 16-17 to include such subject matter, comprising at least a first medical device and a second medical device. The first medical device includes a stimulus circuit configured to deliver electrical pacing therapy to a subject when operatively coupled to a plurality of electrodes, a heart sound sensing circuit configured to produce a sensed heart sound signal representative of mechanical cardiac activity, a control circuit operatively coupled to the stimulus circuit and configured to vary a paced heart rate of the subject according to a specified paced heart rate protocol, and a first communication circuit configured to transfer information wirelessly. The second medical device includes a second communication circuit configured to receive heart sound information from the first medical device, a user interface including a display, and a signal processing circuit. The signal processing circuit is configured to obtain measures of a heart sound parameter as a function of heart rate using the sensed heart sound signal, determine a critical heart rate for the subject using the obtained measures of the heart sound parameter, and present information related to heart rate criticality using the display.

In Example 32, the subject matter of Example 31 optionally includes a signal processing circuit configured to present a graphical plot of measured values of the heart sound parameter as a function of heart rate using the display and indicate the determined critical heart rate in association with the graphical plot.

In Example 33, the subject matter of one or both of Examples 31 and 32 optionally includes a signal processing circuit configured to present, using the display, a graphical plot of values of the heart sound parameter measured as a function of heart rate in ambulatory settings.

In Example 34, the subject matter of one or any combination of Examples 31-33 is optionally configured to obtain measures of S1 heart sound amplitude as the heart sound parameter and identify the critical heart rate as a heart rate corresponding to a maximum measured S1 heart sound amplitude.

In Example 35, the subject matter of one or any combination of Examples 31-34 optionally includes a first medical device including a cardiac signal sensing circuit configured to produce a sensed cardiac signal representative of cardiac depolarization of the subject when coupled electrically to the plurality of electrodes. The signal processing circuit of the second medical device is optionally configured to receive cardiac depolarization information from the first medical device, obtain measures of a systolic time interval (STI) ratio as the heart sound parameter using the heart sound information and the cardiac depolarization information, and identify the critical heart rate as a heart rate corresponding to a minimum measured STI ratio.

These examples can be combined in any permutation or combination. This section is intended to provide a brief overview of subject matter of the present patent application. It is not intended to provide an exclusive or exhaustive explanation of the invention. The detailed description is included to provide further information about the present patent application such as a discussion of the dependent claims and the interrelation of the dependent and independent claims in addition to the statements made in this section.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, the various examples discussed in the present document.

FIG. 1 is an illustration of an example of portions of a system that includes an IMD.

FIG. 2 is an illustration of portions of another system that uses an IMD.

FIG. 3 is a flow diagram of an example of a method of operating a medical device.

FIG. 4 is a graph of the amplitude of the S1 heart sound versus heart rate.

FIG. 5 is a block diagram of portions of an example of a medical device.

FIG. 6 is an illustration of an electrocardiogram and a phonocardiogram.

FIG. 7 is an illustration of relative occurrence of fiducials in a sensed cardiac signal and a sensed heart sound signal.

FIG. 8 is a graph of systolic time interval ratio versus heart rate.

FIG. 9 is a block diagram of portions of an example of a medical device system.

### DETAILED DESCRIPTION

An ambulatory medical device can include one or more of the features, structures, methods, or combinations thereof described herein. For example, a cardiac monitor or a cardiac stimulator may be implemented to include one or more of the advantageous features or processes described below. It is intended that such a monitor, stimulator, or other implantable or partially implantable device need not include all of the features described herein, but may be implemented to include selected features that provide for unique structures or functionality. Such a device may be implemented to provide a variety of therapeutic or diagnostic functions.

As explained previously, ambulatory pacing therapy should be optimized for a patient. However, evidence-based guidance is generally lacking for physicians to use in selecting or configuring certain pacing configurations versus others for a specific patient. Some physicians decide on a pacing configuration based on experience from trial and error or based on subjective judgment, while most physicians prefer to leave the medical device at the nominal settings due to lack of evidence-based guidance to change the settings from nominal.

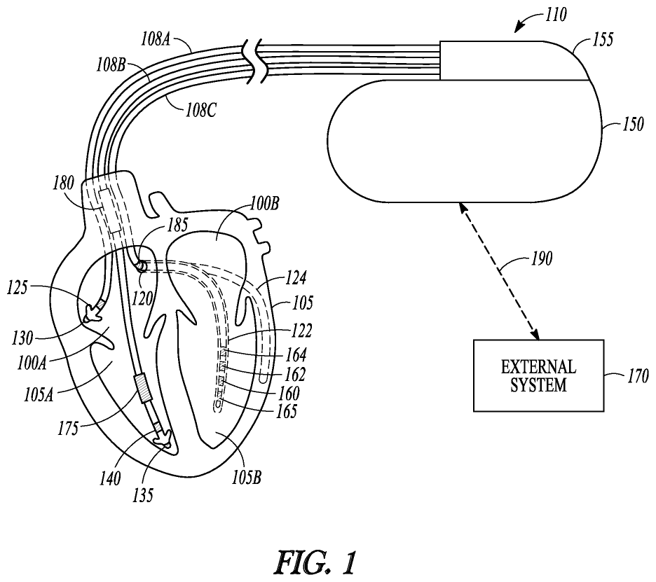

FIG. 1 is an illustration of portions of a system that uses an 1 MB 110. Examples of IMD 110 include, without limitation, a pacemaker, a defibrillator, a cardiac resynchronization therapy (CRT) device, or a combination of such devices. The system also typically includes an IMD programmer or other external device 170 that communicates wireless signals 190 with the IMD 110, such as by using radio frequency (RF) or other telemetry signals.

The 1 MB 110 can be coupled by one or more leads 108A-C to heart 105. Cardiac leads 108A-C include a proximal end that is coupled to IMD 110 and a distal end, coupled by electrical contacts or “electrodes” to one or more portions of a heart 105. The electrodes typically deliver cardioversion, defibrillation, pacing, or resynchronization therapy, or combinations thereof to at least one chamber of the heart 105. The electrodes may be electrically coupled to sense amplifiers to sense electrical cardiac signals. Sensed electrical cardiac signals can be sampled to create an electrogram. An electrogram can be analyzed by the IMD and/or can be stored in the IMD and later communicated to an external device where the sampled signals can be displayed for analysis.

Heart 105 includes a right atrium 100A, a left atrium 100B, a right ventricle 105A, a left ventricle 105B, and a coronary sinus 120 extending from right atrium 100A. Right atrial (RA) lead 108A includes electrodes (electrical contacts, such as ring electrode 125 and tip electrode 130) disposed in an atrium 100A of heart 105 for sensing signals, or delivering pacing therapy, or both, to the atrium 100A.

Right ventricular (RV) lead 108B includes one or more electrodes, such as tip electrode 135 and ring electrode 140, for sensing signals, delivering pacing therapy, or both sensing signals and delivering pacing therapy. Lead 108B optionally also includes additional electrodes, such as for delivering atrial cardioversion, atrial defibrillation, ventricular cardioversion, ventricular defibrillation, or combinations thereof to heart 105. Such electrodes typically have larger surface areas than pacing electrodes to handle the larger energies involved in defibrillation. Lead 108B optionally provides resynchronization therapy to the heart 105. Resynchronization therapy is typically delivered to the ventricles in order to better synchronize the timing of depolarizations between ventricles.

Lead 108B can include a first defibrillation coil electrode 175 located proximal to tip and ring electrodes 135, 140 for placement in a right ventricle, and a second defibrillation coil electrode 180 located proximal to the first defibrillation coil 175, tip electrode 135, and ring electrode 140 for placement in the superior vena cava (SVC). In some examples, high-energy shock therapy is delivered from the first or RV coil 175 to the second or SVC coil 180. In some examples, the SVC coil 180 is electrically tied to an electrode formed on the hermetically-sealed IMD housing or can 150. This improves defibrillation by delivering current from the RV coil 175 more uniformly over the ventricular myocardium. In some examples, the therapy is delivered from the RV coil 175 only to the electrode formed on the 1 MB can 150. In some examples, the coil electrodes 175, 180 are used in combination with other electrodes for sensing signals.

The 1 MB 110 can include a third cardiac lead 108C attached to the 1 MB 110 through the header 155. The third cardiac lead 108C includes electrodes 160, 162, 164, and 165 placed in a coronary vein lying epicardially on the left ventricle (LV) 105B via the coronary vein. The third cardiac lead 108C may include anywhere from two to eight electrodes and may include a ring electrode 185 positioned near the coronary sinus (CS) 120.

Note that although a specific arrangement of leads and electrodes are shown the illustration, an IMD can be configured with a variety of electrode arrangements, including transvenous, endocardial, and epicardial electrodes (i.e., intrathoracic electrodes), and/or subcutaneous, non-intrathoracic electrodes, including can, header, and indifferent electrodes, and subcutaneous array or lead electrodes (i.e., non-intrathoracic electrodes). The present methods and systems will work in a variety of configurations and with a variety of electrodes. Other forms of electrodes include a mesh including multiple electrodes, patch electrodes, and wireless electrostimulation nodes or “seeds” which can be applied to portions of heart 105. Some IMDs are leadless, such as insertable cardiac monitors (ICMs) and leadless cardiac pacemakers (LCPs) for example. Leadless IMDs may include two or more electrodes on the housing and/or header of the device to sense the electrical signals of the heart.

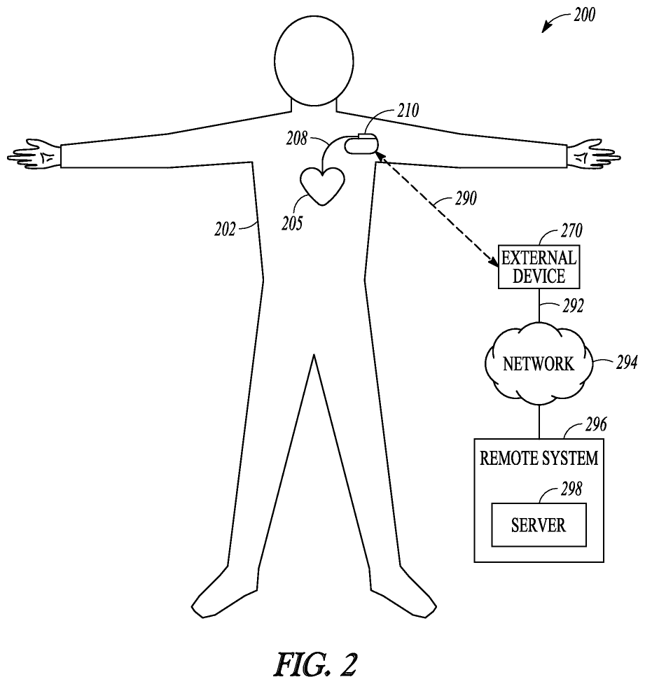

FIG. 2 is an illustration of portions of another system 200 that uses an IMD 210 to provide a therapy to a patient 202. The system 200 typically includes an external device 270 that communicates with a remote system 296 via a network 294. The network 294 can be a communication network such as a phone network or a computer network (e.g., the internet). In some examples, the external device includes a repeater and communicated via the network using a link 292 that may be wired or wireless. In some examples, the remote system 296 provides patient management functions and may include one or more servers 298 to perform the functions.

Some patients (e.g., patients with heart failure) prescribed medical devices exhibit chronotropic incompetence and their heart rate does not increase with increased physical activity or other increased demand on their cardiorespiratory system. For these patients, adaptive rate pacing therapy can be used to sense changes in demand on the cardiorespiratory system and adjust heart rate accordingly. For example, some ambulatory medical devices can include a physical activity sensor (e.g., an accelerometer) or a respiratory minute ventilation sensor (e.g., a thoracic impedance sensor). The output signal from these sensors can provide information related to the level of activity-related metabolic demand of the patient, and the ambulatory medical device may change the paced heart rate (or a conversely change the pacing interval) in response to changes in activity-related metabolic demand indicated by the output signal.

For a situation where the physical activity sensor indicates that the physical activity level of a patient is sustained, the ambulatory medical device may increase the paced heart rate of the patient to a maximum heart rate limit. This is sometimes referred to as a maximum sensor rate (MSR). A common tool used to decide the setting for the maximum heart rate limit is the age-predicted maximum of 220 beats per minute (220 bpm) minus the age of the patient. However, there can be wide variability for the optimum maximum heart rate limit among patients.

For those with normal cardiovascular systems there is a relationship between heart rate and cardiac contractility. This relationship is sometimes referred to as a force-frequency response (FFR). Cardiac contractility increases with increased heart rate until a critical heart rate. Beyond the critical heart rate, cardiac contractility decreases with increasing heart rate. For patients with cardiac disease such as heart failure, the FFR is blunted but a critical heart rate still exists for the patients. It would be optimum for these patients to have the maximum heart rate limit for the ambulatory medical device set to the critical heart rate of the patient.

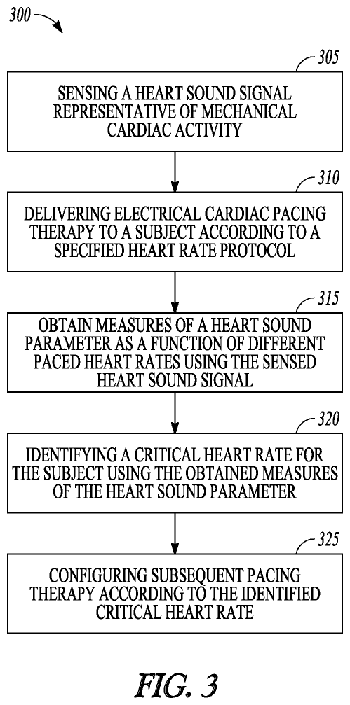

FIG. 3 is a flow diagram of an example of an automated method 300 of operation of a medical device or medical device system. At 305, a heart sound signal is sensed. Heart sounds are associated with mechanical vibrations from activity of a patient's heart and the flow of blood through the heart, and the sensed heart sound signal is representative of mechanical cardiac activity of a subject. Heart sounds recur with each cardiac cycle and are separated and classified according to the activity associated with the vibration. The first heart sound (S1) is associated with systole and is the vibrational sound made by the heart during the near simultaneous closure of the mitral and tricuspid valves. The second heart sound (S2) marks the end of systole and the beginning of diastole. The third heart sound (S3) and fourth heart sound (S4) are related to filling pressures of the left ventricle during diastole. The sensed heart sound signal includes a representation of one or more of the heart sounds. Heart sounds can be sensed using a microphone or an accelerometer. They can be sensed intra-cardiac (endocardial accelerations), intrabody or externally (e.g., using a patch or other wearable device). Heart sounds can also be referred to as siesmocardiogram.

At 310, electrical cardiac pacing therapy is delivered to the subject. The pacing therapy is delivered according to a specified paced heart rate protocol in which the paced heart rate is varied. For example, the paced rate may be increased according to a ramp-up protocol. Conversely, the paced interval between pulses (e.g., a paced ventricular interval, or V-V interval) is reduced according to the ramp-up protocol.

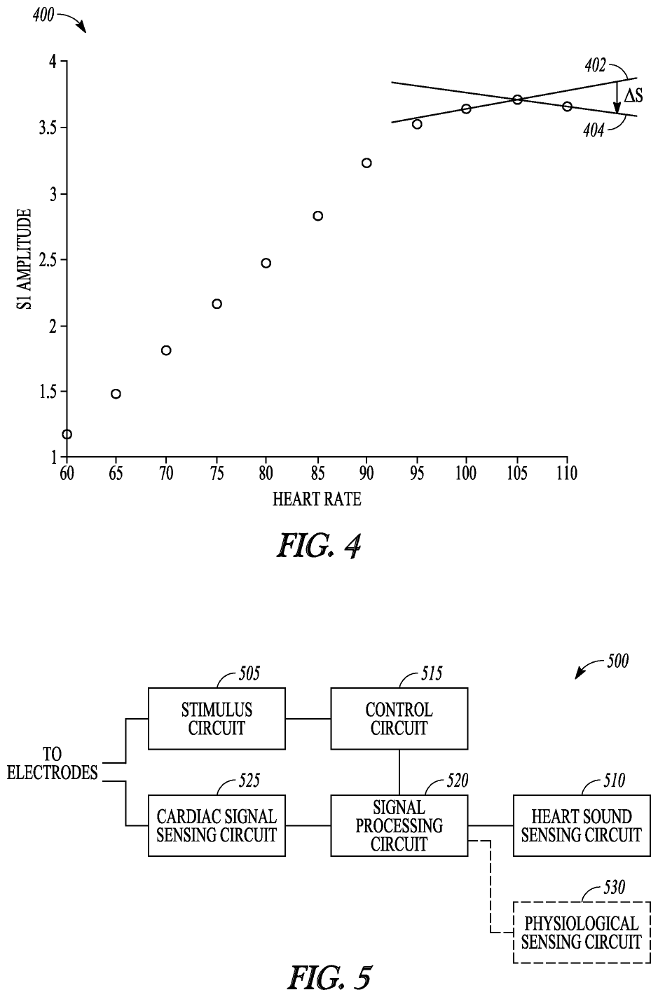

At 315, measures of a heart sound parameter as a function of different paced heart rates are obtained using the sensed heart sound signal. In some examples, the amplitude of the S1 heart sound versus heart rate is measured. FIG. 4 is a graph 400 of S1 amplitude versus heart rate. The graph or curve can be determined by the medical device system. The horizontal axis is heart rate in beats per minute (bpm) and the vertical axis may be in volts of the sensed heart sound signal. Another example of a measure of a heart sound parameter is a systolic time interval (STI) ratio as described below.

At 320, a critical heart rate for the subject is determined using the obtained measures of the heart sound parameter. The amplitude of the S1 heart sound can be a proxy measurement for ventricular contractility and can be used to determine the critical heart rate for a particular patient or subject. As explained previously herein, cardiac contractility decreases with increasing heart rate beyond the critical heart rate. It can be seen in the graph 400 of FIG. 4 that the S1 heart sound amplitude increases with heart rate to about 105 beats per minute (105 bpm) and then begins to decrease with increased heart rate. Thus, the medical device system may identify 105 bpm as the critical heart for the example of FIG. 4.

Returning to FIG. 3 at 325, subsequent pacing therapy provided by the medical device system is configured using the identified critical heart rate. For example, based on the graph 400 of FIG. 4, the medical device system may configure the maximum heart rate limit or a maximum pacing rate for the subsequent pacing therapy as 105 bpm, or near 105 bpm. Thus, a physiological optimum parameter is determined for the device to assist a physician in customizing device-based therapy for a particular patient.

FIG. 5 is a block diagram of portions of an example of a medical device 500. The medical device 500 includes a stimulus circuit 505 and a heart sound sensing circuit 510. The stimulus circuit 505 provides electrical stimulation therapy when operatively coupled to electrodes (not shown). In some examples, the stimulus circuit provides electrical therapy to treat bradycardia. The heart sound sensing circuit 510 produces a heart sound signal. Examples of a heart sound sensing circuit 510 include an accelerometer, a strain gauge, and a microphone. The heart sound sensing circuit may be positioned within the hermetic housing of an implantable or insertable device.

The medical device 500 also includes a control circuit 515. The control circuit 515 may include one or more of a microprocessor, a digital signal processor, application specific integrated circuit (ASIC), field programmable gate array (FPGA), or other type of processor, interpreting or executing instructions included in software or firmware. The control circuit 515 is configured (e.g., by programming) to control the delivery of the pacing therapy. The medical device 500 also includes a signal processing circuit 520. The signal processing circuit 520 may be included in the control circuit 515 or may be a separate circuit, such as a separate processor.

The control circuit 515 may control delivery of a specified regimen of pacing therapy in a normal operating mode. To identify a critical heart rate, the control circuit may leave the normal operating mode to vary the paced heart rate according to a specified paced heart rate protocol (e.g., a critical heart rate protocol). The control circuit 515 may increase the paced heart rate according to a ramp-up pacing rate protocol. In some examples, the control circuit 515 may vary the paced heart rate according to a pseudo-random pacing rate protocol. As the heart rate is varied, the signal processing circuit 520 produces measurements of a heart sound parameter as a function of heart rate. The signal processing circuit 520 determines the critical heart rate of the subject using the determined measurements.

According to some examples, the heart sound parameter is the amplitude of the S1 heart sound as in FIG. 4. As explained previously herein, the amplitude of the S1 heart sound can be a proxy measurement for cardiac contractility. The control circuit 515 may start the paced heart rate at 60 bpm and gradually increase the heart rate. Each stage or step of the protocol may pace the heart at the specified rate for two to five minutes. The first few measurements of the S1 amplitude of beats immediately following a step change in rate may be discarded to remove transients from the signal processing analysis. The retained measurements for a particular step may be filtered (e.g., using an ensemble average), and filtered measurements may be used to obtain a reliable heart sound amplitude curve as in FIG. 4.

Different methods may be used by the signal processing circuit 520 to identify the critical rate of the subject. The signal processing circuit 520 may identify the critical rate when the S1 amplitude decreases by a specified threshold (e.g., a specified percentage of the amplitude) as the heart rate is increased. The critical heart rate can be identified as the last paced heart rate before the decrease in the S1 amplitude.

In some examples, the signal processing circuit 520 uses a change in slope of the S1 amplitude curve to identify the critical rate. The signal processing circuit 520 may identify the critical rate when the slope of the S1 vs Heart Rate curve decreases by a specified threshold amount. For instance, FIG. 4 shows a first line 402 determined using points of the S1 amplitude curve and a second line 404. The slopes of the lines may be determined as Aamplitude/Abpm. The slope of the second line decreases from the first line 402 to the second line 404. The signal processing circuit 520 may identify the critical rate as paced rate just before the determined slope changes to less than or equal to zero, or when the decrease in slope ΔS exceeds a threshold amount.

According to some examples, the heart sound parameter is an STI ratio. The STI ratio can also be a proxy measurement for ventricular contractility and can be used to determine the critical heart rate for a particular patient or subject. Returning to FIG. 5, the medical device 500 may include a cardiac signal sensing circuit 525 that produces a sensed cardiac signal representative of cardiac depolarization of the subject when coupled to the electrodes. The signal processing circuit 520 obtains measurements for the STI ratio using the cardiac signal and the heart sound signal.

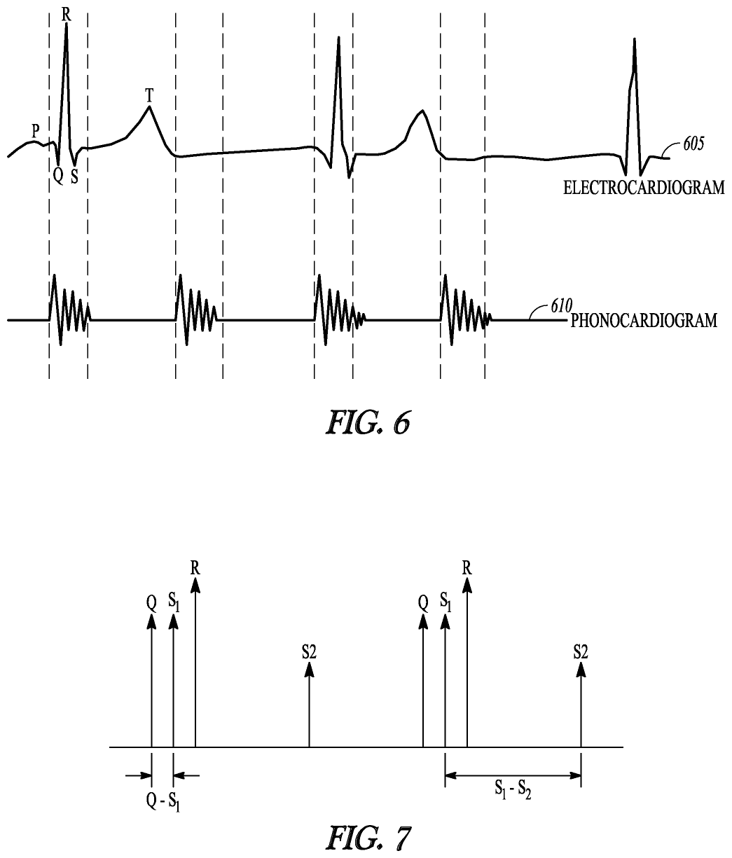

FIG. 6 is an illustration of an electrocardiogram 605 as the sensed cardiac signal and a phonocardiogram 610 as the sensed heart sound signal. The electrocardiogram 605 shows the PQRST signal waveform complex associated with a depolarization. FIG. 7 is an illustration of relative occurrence of fiducials in a sensed cardiac signal and a sensed heart sound signal, including the Q-wave, the onset of the S1 heart sound, the R-wave, and the onset of the S2 heart sound. The STI ratio can include a time interval between a cardiac signal fiducial (e.g., a Q-wave) and the S1 heart sound (Q-S1 interval), and a time interval between the S1 heart sound and an S2 heart sound (S1-S2 interval), or (Q-S1)/(S1-Ω). The Q-S1 interval can be the pre-ejection period (PEP) and S1-S2 interval can be the left ventricular ejection time (LVET), and the STI ratio is PEP/LVET.

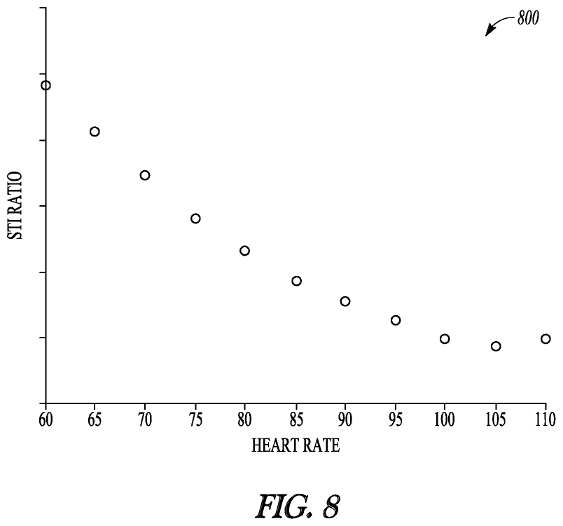

FIG. 8 is a graph 800 of STI ratio versus heart rate in bpm. The graph or curve can be determined by the medical device 500 of FIG. 5. The STI ratio decreases as the heart rate increases indicating an improvement in cardiac contractility. Beyond the critical heart rate, the STI ratio increases indicating that cardiac contractility is no longer improving with the increase in heart rate.

The signal processing circuit 520 may identify the critical heart rate as a heart rate corresponding to a minimum measured STI ratio. It can be seen in the graph 800 of FIG. 8 that the STI ratio decreases with heart rate to about 105 beats per minute (105 bpm) and then begins to increase with increased heart rate. Thus, the medical device may identify 105 bpm as the critical heart as in the example of FIG. 4.

Different methods may be used by the signal processing circuit 520 to identify the critical rate of the subject from the STI ratio. The signal processing circuit 520 may identify the critical rate when the STI ratio increases by a specified threshold (e.g., a specified percentage of the amplitude) as the heart rate is increased. In some examples, the signal processing circuit 520 uses a change in slope of the STI ratio curve to identify the critical rate. In the example curve of FIG. 8, the slope of the STI ratio curve will increase from 105 bpm to 110 bpm. The signal processing circuit 520 may identify the critical rate when the slope of the STI ratio curve increases by a specified threshold amount. In some examples, the signal processing circuit 520 may identify the critical rate for the heart rate at which the slope of the STI ratio curve is zero (i.e., a flat slope); indicating the minimum value of the curve. The heart sound parameter can be a ratio that includes other fiducials of the cardiac signal and the heart sound signal. For instance, the R-wave can be used instead of the Q-wave and the heart sound parameter can be the ratio (S1-R)/(S1-S2).

The medical device 500 of FIG. 5 may also include a physiologic sensing circuit 530 that produces a sensed physiological signal representative of physiological information of a subject. The physiologic sensing circuit 530 may include an accelerometer representative of the level of physical activity of the subject. In another example, the physiologic sensing circuit 530 may be a respiration sensing circuit and the physiological signal is representative of respiration of the subject. The control circuit 515 may provide adaptive rate pacing and change the paced heart rate based on the demand on the cardiorespiratory system as indicated by the sensed physiological signal. The control circuit 515 may set the maximum pacing rate to the critical rate when the critical rate is determined. The control circuit 515 may change the paced heart rate up to the critical heart rate when the demand on the cardiorespiratory system is sustained.

While the embodiments described herein include determining the maximum pacing rate using a critical rate, it is contemplated that other properties extracted from the heart sound and heart rate graph could be used to determine the maximum pacing rate, such as slope, amplitude, derivative, template matching, etc.

The device-based signal processing can perform analyses to uncover changes in the sensed signals undetectable by a clinician that can change the desired critical rate for the patient. Moreover, the measurements can be made while the patient is ambulatory and do not need to be made while the patient is in a clinic. In this way, the critical heart rate can be determined according to a schedule. For instance, medical device may be programmed to determine the critical heart rate at different times of the day. The signal processing circuit 520 may gather paired measures of the heart sound parameter and heart rate during naturally occurring changes in heart rate over a specified time interval. The signal processing circuit may use the paired measures of the heart sound parameter and heart rate to determine the critical heart rate. Different critical heart rates can be used based on time of day. The determined critical heart rate or rates can be later uploaded for inspection by a clinician.

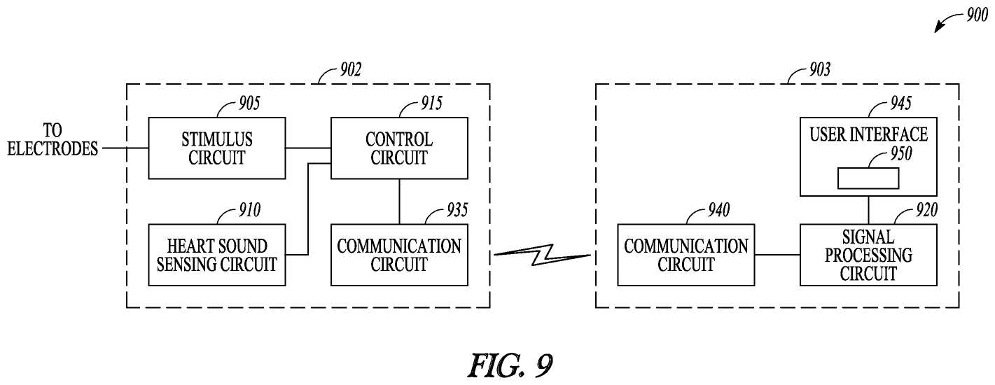

FIG. 9 is a block diagram of portions of an example of a medical device system. The medical device system 900 includes a first medical device 902 and a second medical device 903. The first medical device 902 can be an ambulatory medical device (e.g., an IMD) and the second medical device 903 may be a programmer for the first ambulatory medical device 902. The first medical device 902 includes a stimulus circuit 905, a heart sound sensing circuit 910, and a control circuit 915. The stimulus circuit 905 delivers electrical pacing therapy to a subject when operatively coupled to electrodes. The heart sound sensing circuit 910 produces a sensed heart sound signal representative of mechanical cardiac activity, and the control circuit 915 varies a paced heart rate of the electrical pacing therapy according to a specified paced heart rate protocol. The first medical device 902 also includes a communication circuit 935 to transfer information wirelessly to a separate device.

The second medical device 903 includes a signal processing circuit 920, a user interface 945 including display 950, and a communication circuit 940 to wirelessly receive information such as heart sound information rom the first medical device 902. The signal processing circuit 920 obtains measures of a heart sound parameter as a function of heart rate using the sensed heart sound signal. In some examples, the communication circuit 940 receives a sampled heart sound signal from the first medical device 902 and calculates the heart sound parameter from the received heart sound signal. In some examples, the signal processing circuit 920 receives values of the heart sound parameter for different heart rates from the first medical device 902. The heart sound parameter may include any of the example S1 heart sound parameters described herein.

The signal processing circuit 920 determines the critical heart rate using the obtained measures of the heart sound parameter and presents information related to heart rate criticality using the display. In some examples, the signal processing circuit 920 presents a graphical plot of measured values of the heart sound parameter as a function of heart rate using the display. The graphical plot may include a curve of S1 amplitude as a function of heart rate as in FIG. 4, or the STI ratio as a function of heart rate as in FIG. 8. The signal processing circuit 920 may indicate the determined critical heart rate in association with the graphical plot. For instance, the signal processing circuit 920 may indicate the critical heart rate on the graphical plot or present a value of the critical heart rate. In some examples, the signal processing circuit 920 presents the value of the critical heart to a user (e.g., a clinician) and presents a recommendation to use the critical heart rate as a maximum paced rate. The user can confirm the critical rate as the maximum pacing rate using the user interface and the second medical device may program the first medical device accordingly.

The devices, systems, and methods described herein can provide hemodynamically optimized adaptive rate response pacing using measurements of heart sounds. The force frequency response of the patient determined using proxy measurements performed by the devices, systems, and methods indicates the optimum maximum paced heart rate for the patient.

### ADDITIONAL DESCRIPTION

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like. In some examples, a carrier medium can carry code implementing the methods. The term “carrier medium” can be used to represent carrier waves on which code is transmitted.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An apparatus comprising:
a stimulus circuit configured to deliver electrical pacing therapy to a subject when operatively coupled to a plurality of electrodes;
a heart sound sensing circuit configured to produce a sensed heart sound signal representative of mechanical cardiac activity; and
a signal processing circuit configured to:
a signal processing circuit configured to:
obtain measures of a heart sound parameter as a function of heart rate using the sensed heart sound signal; and
determine a maximum pacing rate using a critical heart rate identified using the obtained measures of the heart sound parameter as a function of heart rate.

2. The apparatus of claim 1, wherein the signal processing circuit is configured to:
obtain measures of S1 heart sound amplitude as the heart sound parameter; and
identify the critical heart rate as a heart rate corresponding to a maximum measured S1 heart sound amplitude.

3. The apparatus of claim 1, including:
a cardiac signal sensing circuit configured to produce a sensed cardiac signal representative of cardiac depolarization of the subject when coupled electrically to the plurality of electrodes;
wherein signal processing circuit is configured to:
obtain measures of a systolic time interval (STI) ratio as the heart sound parameter using the sensed cardiac signal and the sensed heart sound signal; and
identify the critical heart rate as a heart rate corresponding to a minimum measured STI ratio.

4. The apparatus of claim 3, wherein the signal processing circuit is configured to calculate, as the STI ratio, a ratio including a time interval between a cardiac signal fiducial and the S1 heart sound and a time interval between the S1 heart sound and an S2 heart sound.

5. The apparatus of claim 1, wherein the signal processing circuit is configured to:
determine slope of the measures of the heart sound parameter as a function of heart rate; and
identify the critical heart rate as a heart rate corresponding to a minimum determined slope.

6. The apparatus of claim 1, including:
a control circuit operatively coupled to the stimulus circuit and configured to vary a paced heart rate of the subject according to a specified paced heart rate protocol;
wherein the signal processing is configured to collect the measures of the heart sound parameter according to the specified paced heart rate protocol and determine the critical heart rate using the collected measures of the heart sound parameter.

7. The apparatus of claim 6, wherein the control circuit is configured to vary the paced heart rate according to one of a ramp-up pacing rate protocol or a pseudo-random pacing rate protocol.

8. The apparatus of claim 6, including:
a physiologic sensing circuit configured to produce a sensed physiological signal representative of physiological information of the subject;
wherein the control circuit is configured to change the paced heart rate up to a maximum heart rate limit according to the sensed physiological signal, and set the maximum heart rate limit to the determined critical heart rate.

9. The apparatus of claim 1, wherein the signal processing circuit is configured to gather paired measures of the heart sound parameter and heart rate during naturally occurring changes in heart rate over a specified time interval and use the paired measures of the heart sound parameter and heart rate to determine the critical heart rate.

10. The apparatus of claim 1, wherein the signal processing circuit is configured to filter measures of the heart sound parameter after a change in heart rate and use filtered measures of the heart sound parameter as a function of heart rate to determine the critical rate.

11. An automated method of operation of a medical device system, the method comprising:
sensing a heart sound signal representative of mechanical cardiac activity of a subject;
delivering electrical cardiac pacing therapy to the subject, including varying a paced heart rate of the subject according to a specified paced heart rate protocol;
obtaining measures of a heart sound parameter as a function of different paced heart rates using the sensed heart sound signal; and
configuring a maximum pacing rate using a critical heart rate identified using the obtained measures of the heart sound parameter.

12. The method of claim 11,
wherein the obtaining measures of the heart sound parameter includes obtaining measures of S1 heart sound amplitude as the paced heart rate is varied; and
wherein the identifying the critical heart rate includes identifying the critical heart rate as a heart rate corresponding to a maximum measured S1 heart sound amplitude.

13. The method of claim 11, including:
producing a sensed cardiac signal representative of cardiac depolarization of the subject;
wherein the obtaining measures of the heart sound parameter includes obtaining measures of a systolic time interval (STI) ratio using the sensed cardiac signal and the sensed heart sound signal as the paced rate is varied; and
wherein the identifying the critical heart rate includes identifying the critical heart rate as a heart rate corresponding to a minimum measured STI ratio.

14. The method of claim 13, wherein the obtaining measures of the STI ratio includes calculating a ratio including a time interval between a cardiac signal fiducial and the S1 heart sound and a time interval between the S1 heart sound and an S2 heart sound.

15. The method of claim 11, wherein the identifying the critical heart rate includes:
determining a slope of the measures of the heart sound parameter as a function of heart rate; and
identifying the critical heart rate as a heart rate corresponding to a minimum determined slope.

16. A medical device system comprising:
a first medical device including:
a stimulus circuit configured to deliver electrical pacing therapy to a subject when operatively coupled to a plurality of electrodes;
a heart sound sensing circuit configured to produce a sensed heart sound signal representative of mechanical cardiac activity;
a control circuit operatively coupled to the stimulus circuit and configured to vary a paced heart rate of the subject according to a specified paced heart rate protocol; and
a first communication circuit configured to transfer information wirelessly; and
a second medical device including:
a second communication circuit configured to receive heart sound information from the first medical device;
a user interface including a display; and
a signal processing circuit configured to:
obtain measures of a heart sound parameter as a function of heart rate using the sensed heart sound signal;
determine a critical heart rate for the subject using the obtained measures of the heart sound parameter; and
present information related to heart rate criticality using the display.

17. The medical device system of claim 16, wherein the signal processing circuit is configured to present a graphical plot of measured values of the heart sound parameter as a function of heart rate using the display, and indicate the determined critical heart rate in association with the graphical plot.

18. The medical device system of claim 16, wherein the signal processing circuit is configured to present, using the display, a graphical plot of values of the heart sound parameter measured as a function of heart rate in ambulatory settings.

19. The medical device system of claim 16, wherein signal processing circuit is configured to:
obtain measures of S1 heart sound amplitude as the heart sound parameter; and
identify the critical heart rate as a heart rate corresponding to a maximum measured S1 heart sound amplitude.

20. The medical device system of claim 16,
wherein the first medical device includes:
a cardiac signal sensing circuit configured to produce a sensed cardiac signal representative of cardiac depolarization of the subject when coupled electrically to the plurality of electrodes;
wherein signal processing circuit is configured to:
receive cardiac depolarization information from the first medical device;
obtain measures of a systolic time interval (STI) ratio as the heart sound parameter using the heart sound information and the cardiac depolarization information; and
identify the critical heart rate as a heart rate corresponding to a minimum measured STI ratio.
